(19) United States
(12) Patent Application Publication
(10) Pub. No.: US 2023/0105487 A1
(43) Pub. Date: Apr. 6, 2023

(54) METHODS AND APPARATUS FOR OBTAINING FINE-GRAINED GLEASON GRADES AND PREDICTING CLINICAL OUTCOMES IN PROSTATE CANCER

(71) Applicant: AIRAMATRIX PRIVATE LIMITED, Mumbai (IN)

(72) Inventors: Nitin SINGHAL, Bangalore (IN); Nilanjan CHATTOPADHYAY, Gurgaon (IN)

(73) Assignee: AIRAMATRIX PRIVATE LIMITED, Mumbai (IN)

(21) Appl. No.: 17/961,336

(22) Filed: Oct. 6, 2022

(30) Foreign Application Priority Data
Oct. 5, 2021 (IN) 202121045155

(51) Int. Cl. G16H 50/30

(52) U.S. Cl. CPC G16H 50/30

(57) ABSTRACT

Embodiments herein relate to prostate cancer management, and more particularly to managing prostate cancer by obtaining fine-grained Gleason grades and predicting clinical outcomes from needle core biopsy images and radical prostatectomy images. A deep learning model is fed with a plurality of training images and is trained to recognize fine-grained Gleason patterns in the plurality of training images. The output of the deep learning model is representative of the fine-grained Gleason score.

### CROSS REFERENCE TO RELATED APPLICATION

This application is based on and derives the benefit of Indian Provisional Application, 202121045155, the contents of which are incorporated herein by reference.

### TECHNICAL FIELD

Embodiments disclosed herein relate to prostate cancer, and more particularly to managing prostate cancer by using a deep learning method to obtain fine-grained Gleason grades and predict clinical outcomes in prostate cancer.

### BACKGROUND

The second most frequent cancer diagnosis made in men is prostate cancer and is the fifth leading cause of death worldwide. There are approximately 1.5 million new cases of prostate cancer reported annually, with a higher prevalence in the developed countries. Differences in the incidence rates worldwide can be caused by differences in the use of diagnostic testing. Prostate cancer affects one in every eight men at some point in their lives, yet the severity and prognosis of the disease can vary widely from person to person.

The broad disease spectrum of prostate cancer can range from “indolent and slow growing” to “aggressive and lethal.” Early detection of prostate cancer is possible, but early determination of the severity of the prostate cancer is not, leading to uncertainty on the level of disease progression. As such, an error in diagnosis could lead to an overtreatment with significant morbidity. It can be difficult to treat prostate cancer clinically because of its proliferation throughout the body, particularly to the bones. Clinical management can be complicated by the disease's wide range of manifestations.

Current methods for treating prostate cancer rely on risk stratification, where the severity of the prostate cancer or the risk it poses to the affected individual is determined. Gleason grading by a pathologist is the gold-standard in risk stratification of patients. The pathologist looks at the growth pattern, which can involve the microscopic arrangement, architecture, or pattern of glands in the prostate cancer sample, and assigns a grade that represents the severity of the prostate cancer or the risk associated with it. The current Gleason grading system assigns a grade (3, 4, or 5) for each growth pattern (primary and secondary growth pattern), and depending on the grade for each growth pattern, the Gleason grading system categorizes prostate cancer risk categories based on the Gleason scores. The scores can range from 3+3 (low risk) to 5+5 (high risk), where the first integer grade represents the severity of the primary growth pattern (most predominant) and the second integer represents the severity of the secondary growth pattern, as determined by histological evaluation by the pathologist. This grading system may have five prognostically distinct grade groups—group 1 (scores of 3+3 and below), group 2 (score of 3+4), group 3 (score of 4+3), group 4 (score of 3+5, 5+3, and 4+4) and group 5 (score of 4+5, 5+4, and 5+5).

FIG. 1 illustrates the traditional Gleason grading system. Grade 1 is assigned for a growth pattern having glands that are small and uniform. Grade 2 is assigned for a growth pattern having glands that have more stroma. Grade 3 is assigned for a growth pattern having glands that have distinctly infiltrative margins. Grade 4 is assigned for a growth pattern having irregular masses of neoplastic glands. Grade 5 is assigned for a growth pattern having occasional gland formation. The severity of the prostate cancer can be determined by its assigned grade, wherein the severity increases from Grade 1 to Grade 5, with Grade 1 having the lowest severity or risk and Grade 5 having the highest severity or risk.

However, the Gleason grading system has several limitations, as it assigns integer values for a growth pattern, when instead it would be beneficial to have assign a continuous grade to the growth pattern that would provide a more refined risk classification of the prostate cancer. For example, a continuous grade such as 3.7 would be categorized as grade 3 in the traditional Gleason grading system, which would misrepresent the actual severity and risk of prostate cancer. Additionally, as the Gleason grading is done through a microscopic evaluation conducted by a pathologist, it can be subjective in nature and have a low reproducibility.

If individuals undergo radical prostatectomy for treating their prostate cancer, they may be susceptible to a biochemical recurrence and metastasis risk, which significantly impacts their overall survival. Therefore, having a way to determine the biochemical recurrence and metastasis risk could be lifesaving.

### OBJECTS

The principal object of embodiments herein is to disclose methods and apparatus for obtaining fine-grained Gleason grades and predicting clinical outcomes in prostate cancer, which enables prediction of disease progression for accurate risk stratification to enable patient specific therapy regimens, and prevent treatment-related mortality.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, throughout which like reference letters indicate corresponding parts in the various figures. The embodiments herein will be better understood from the following description with reference to the drawings, in which:

FIG. 1 illustrates the traditional Gleason grading system, according to prior arts;

FIG. 2 illustrates a comparison between the traditional Gleason grade and a fine-grained Gleason grade, according to embodiments as disclosed herein;

FIG. 3 illustrates three SegFormer networks that are trained to identify fine-grained Gleason patterns at various magnification levels, according to embodiments as disclosed herein;

FIGS. 4A and 4B depict example workflows, according to embodiments as disclosed herein;

FIG. 5 depicts example tissues belonging to a plurality of grades in the fine-grained Gleason grading system, according to embodiments as disclosed herein;

FIG. 6 illustrates a method for producing a fine-grained Gleason grade, according to embodiments as disclosed herein;

FIG. 7 illustrates a comparison of the accuracy between a model using Cancer of the Prostate Risk Assessment Postsurgical (CAPRA-S) and a model using fine-grained Gleason grades to determine the risk of prostate cancer recurrence and metastasis, according to embodiments as disclosed herein; and

FIG. 8 illustrates a computing device that is configured to produce a fine-grained Gleason grade, according to embodiments as disclosed herein.

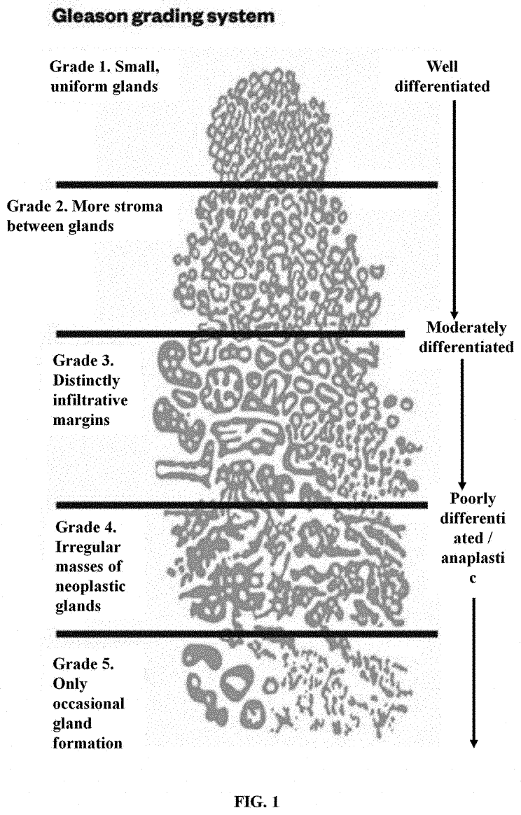

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein achieve methods and systems for obtaining fine-grained Gleason grades and predicting clinical outcomes in prostate cancer, which enables prediction of disease progression for accurate risk stratification to enable patient specific therapy regimens, and prevent treatment-related mortality. A fine-grained Gleason grade can be a continuous value (at least one decimal place) instead of an integer value. For example, for an integer value for a growth pattern can be 3, whereas a continuous value for the same growth pattern can be 3.3 or 3.34 or 3.347.

The embodiments herein use a deep learning method for fine-grained Gleason pattern estimation. Some of the practical applications for this can be predicting the metastasis risk by correlating the percentages of fine-grained Gleason grades on prostatectomy cases with metastasis risk, and predicting the metastasis risk by correlating the percentages of fine-grained Gleason grades on needle core biopsies. Another application could be to correlate the percentages of fine-grained Gleason grades on prostatectomy cases with biochemical recurrence risk.

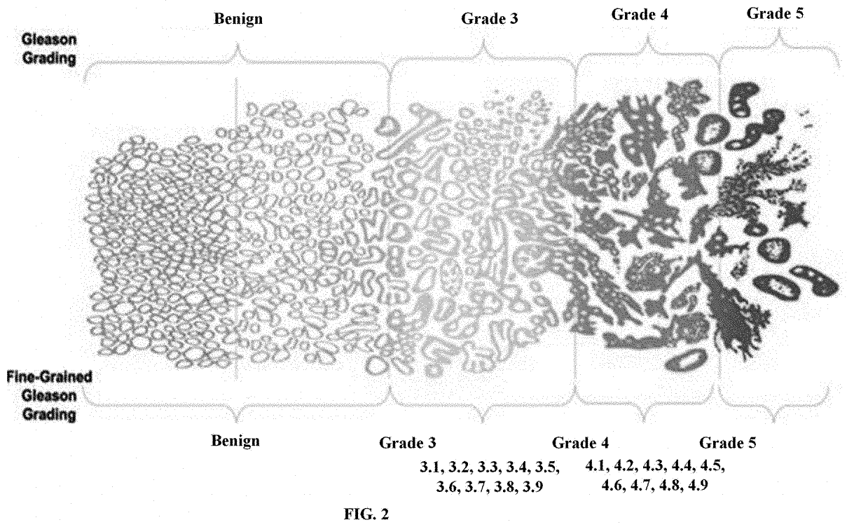

Referring now to the drawings, and more particularly to FIGS. 2 through 8, where similar reference characters denote corresponding features consistently throughout the figures, there are shown embodiments.

FIG. 2 illustrates a comparison between the traditional Gleason grade and the fine-grained Gleason grade, according to embodiments as disclosed herein. For growth patterns that would be assigned a grade of integer value 3 or 4 under the traditional Gleason grading system, the embodiments disclosed herein would assign continuous values to the same growth patterns. The continuous vales include decimal points that are more indicative of the severity of the risk of the prostate cancer than just integer values alone. For example, a growth pattern in the traditional Gleason grading system that receives a grade 3 could receive a fine-grained Gleason grade of 3.9 for the same growth pattern. As the growth pattern having the grade 3.9 is leaning more towards grade 4 rather than grade 3, this fine-grained Gleason grade is more reliable in estimating the risk of prostate cancer, as the traditional Gleason grading system would have graded this growth pattern at 3, which would have been misleading with respect to the severity and risk of the prostate cancer.

A deep learning model 110 can be used to recognize and quantify fine-grained Gleason patterns on needle core biopsy and radical prostatectomy specimens on each individual pixel in a whole slide image. The deep learning model 110 may perform a supervised machine learning task of multi label classification. This can allow the assignment of probabilities or likelihoods of the growth patterns in a whole slide image to different grades such as 3, 4, and 5. Using multi label classification over multi class classification has the advantage in not being limited to grading of a growth pattern to only one of 3, 4, or 5.

Each pixel of the whole slide image may be received by the input layer, and after processing by the hidden layers, the output layer of the deep learning model 110 may return a plurality of logits. The plurality of logits may be converted to its corresponding probability value by using a Sigmoid Activation Function (also referred to herein as sigmoid function). The sigmoid function can be used to implement logistic regression and simple neural networks; it can be used as a classifier when there are multiple correct answers to a problem (an example of this situation is multi label classification). The sigmoid function can return a probability value associated with each logit, wherein the probability values returned by the sigmoid function are generally between 0 and 1 or −1 and 1. As the probability values returned by the sigmoid function do not belong to a probability distribution, the sum of the returned values may not add up to 1.

The returned probability values may indicate a probability value for the Gleason pattern belonging to each score (3, 4, or 5). Each channel in the deep learning model 110 may be analyzed independently using the corresponding ground truth (the integer values for the Gleason patterns in the training dataset). To turn the predictions from the deep learning model 110 into a continuous scale, the probabilities may be combined using the following formulas:

$$z = f ( x . w ) = f ( {\sum }_{i = 1}^{n} {x}_{i} {w}_{i} )$$

$${p}_{k} = sigmoid ( {z}_{k} ) , k = \{ 3 , 4 , 5 \}$$

$$S = arg max ( {p}_{k} )$$

$${S}_{CFG} = S + λ ⌊ α \sum {p}_{S +} - β \sum {p}_{S -} ⌉$$

$${{S}_{FG} = S + λ ⌊ α \sum {p}_{S +} - β \sum {p}_{S -} ⌉}_{t}$$

$${{S}_{Upgrade} = ⌊ S + λ ( α \sum {P}_{S +} - β \sum {P}_{S -} ) ⌉}_{i}$$

Here: z is the output (the logits) of the last layer of the CNN model 110; xᵢ is the input for pixel number i; wᵢ is the weight of the corresponding channel; pₖ is the probability of the Gleason pattern in the pixel belonging to a Gleason grade of k, wherein the value of k is 3, 4, or 5, and wherein pₖ is obtained by applying the sigmoid function to zₖ; S is the predicted base class (the ground truth); pₛ₊ is the probability of the Gleason score being higher than the predicted base class (for example, if S is 4, then pₛ₊ is the probability of the grade of the Gleason pattern being higher than 4; pₛ₋ is the probability of the Gleason grade being less than the predicted base class (for example, if S is 4, then pₛ₋ is the probability that the Gleason grade is lesser than 4); S_{CFG} is a continuous fine-grained Gleason grade, wherein the fine-grained Gleason grade can have n number of decimal places; α and β are hyperparameters that may be obtained experimentally; S_{FG} is the final fine-grained Gleason grade that is rounded to its tenth decimal place, where └ ┐ₜ is the tenth decimal place rounding; S_{Upgrade} is an upgraded Gleason grade that is rounded to its integer value, where └ ┐ᵢ is the integer rounding function; λ is equal to zero for the traditional Gleason grading, and λ is equal to 1 for the fine grained Gleason grading and upgraded Gleason grading.

An example of the deep learning model 110 that may be used to compute the fine-grained Gleason grade can be a SegFormer. The SegFormer has a transformer and a multilayer perceptron (MLP) decoder. It can employ a hierarchical transformer encoder like SwinTransformer, but does not employ positional encoding. Because the encoder is hierarchial, the network may generate multi-level multi-scale features with high-resolution coarse features and low-resolution fine-grained features. The lack of positional encoding can allow for applying the model on patches of varying resolutions without significantly degrading performance. The MLP decoder may be lightweight and efficient, capable of producing powerful representations, while remaining simple and computationally demanding.

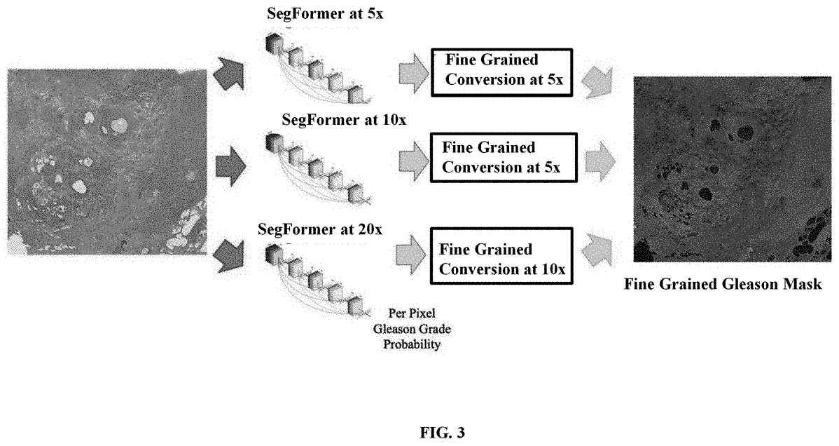

FIG. 3 illustrates the training of three SegFormers at three distinct magnification levels to compute the fine-grained Gleason grade, according to embodiments as disclosed herein. The SegFormers were trained at different magnifications to increase their performance and exploit the hierarchical nature of whole slide images. The whole slide images fed to the SegFormers may have a pathologist's annotations (annotation masks). The final estimated probability for each output channel may be calculated by averaging the anticipated probabilities from the three separate SegFormers at varying magnifications. The three magnifications that were used were 5×, 10×, and 20×. The resultant probabilities may then be utilized to compute finer Gleason Grade estimates for each pixel.

The SegFormer may already be pretrained on Common Objects in Context (COCO) by default. It may be optimal to train the model with random weights as obtaining segmentation annotations for histopathology images can be a difficult and time-consuming task. Even if annotations are not available, hematoxylin and eosin (H&E) stained whole slide images can be collected. The pretrained weights of the SegFormer model can train the decoder on the H&E whole slide images using Decoder Denoising Pretraining (DDP) protocol. Unlike traditional denoising autoencoder training, in which the entire model is trained for the denoising task, only the decoder may be trained by predicting the noise vector and freezing the encoder. The Dice Score may be significantly improved by using diffusion probabilistic models for image synthesis for pretraining decoder weights of SegFormer. When the number of labelled whole slide images are limited, the improvement may be greater.

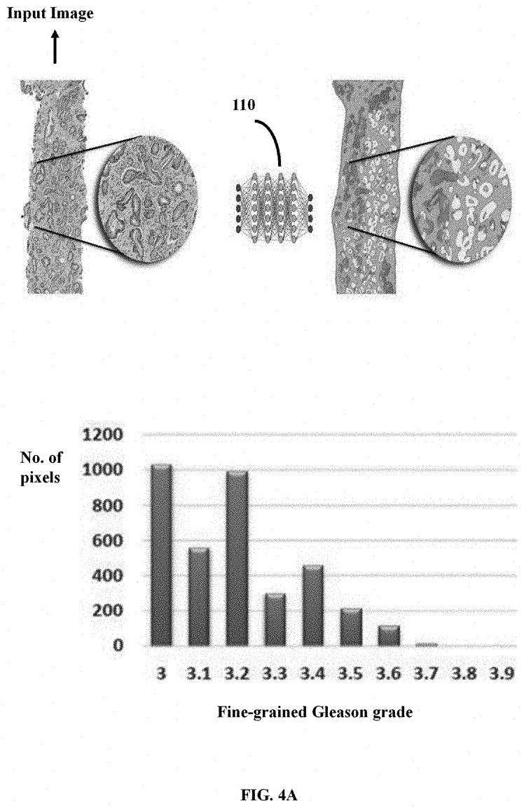

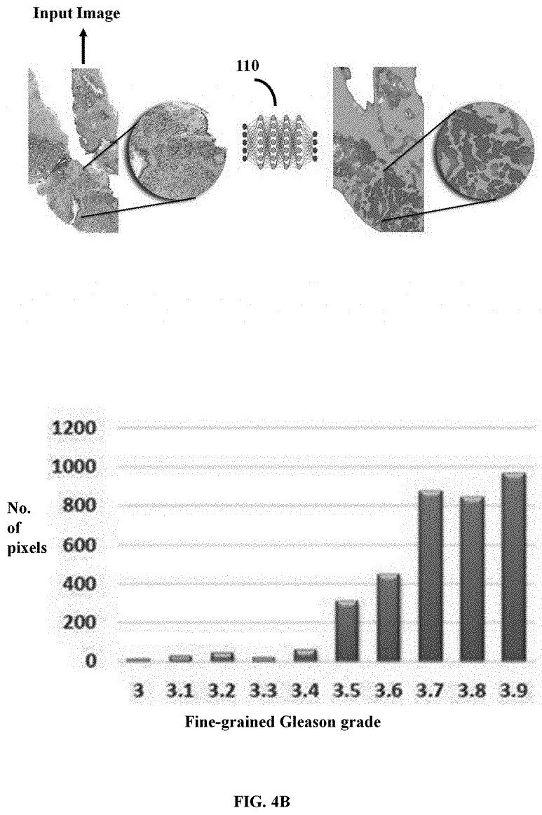

FIGS. 4A and 4B illustrate example workflows, according to embodiments as disclosed herein. In FIG. 4A, the deep learning model 110 may determine the probability of the Gleason grades for the Gleason patterns in each pixel in an input image. Based on this, the fine-grained Gleason grade can be computed for each pixel in the input image. Once the fine-grained Gleason grade for each pixel has been determined, the number of pixels having a certain fine-grained Gleason score can be determined. The graph in FIG. 4A indicates that the majority of pixels in the input image have a fine-grained Gleason grade that is lower than 3.5. Based on this, one can make a prediction that the risk of prostate cancer is similar to risk associated with the traditional Gleason grade of 3.

In FIG. 4B, the graph indicates that a majority of pixels have a fine-grained Gleason grade that is higher than 3.5. Based on this, one can make a prediction that the risk of prostate cancer is similar to the risk associated with the traditional Gleason grade of 4. The example workflow in FIG. 4B further highlights the advantage of the embodiments herein over the traditional Gleason grading system, as in the traditional Gleason grading system the scored would be that of 3, which would be misleading.

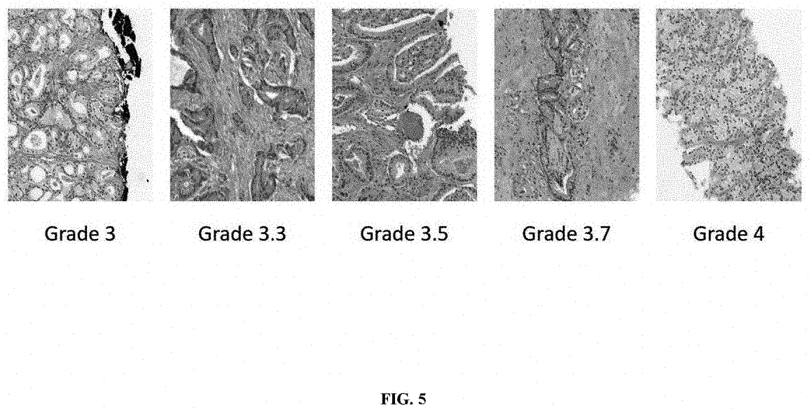

FIG. 5 depicts example tissues belonging to a plurality of grades in the fine-grained Gleason grading system, according to embodiments as disclosed herein. Each region in the tissue was assigned a fine-grained Gleason grade that was more representative of the risk or severity of the prostate cancer.

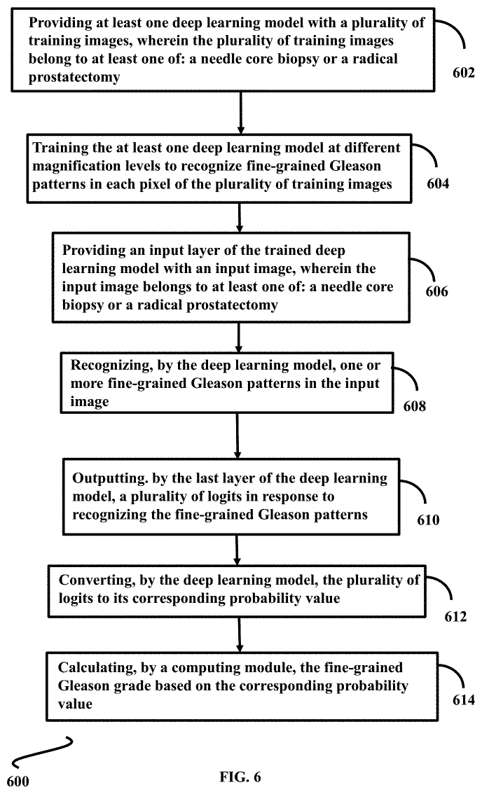

FIG. 6 illustrates a methodology 600 for obtaining a fine-grained Gleason grade, according to embodiments as disclosed herein.

At step 602, a plurality of training images may be fed to at least one deep learning model 110, wherein the plurality of training images belong to at least one of: a needle core biopsy and/or a radical prostatectomy. The deep learning model 110 can be a SegFormer, but is not limited to this.

At step 604, the at least one deep learning model 110 may be trained at different levels of magnification to recognize fine-grained Gleason patterns in each pixel of the plurality of training images.

At step 606, upon training the deep learning model 110, an input image may be fed to the trained deep learning model 110, wherein the input image belongs to at least one of: a needle core biopsy and/or a radical prostatectomy.

At step 608, the trained deep learning model 110 may recognize one or more fine-grained Gleason patterns in the input image.

At step 610, the last output layer of the deep learning model 110 may output a plurality of logits in response to recognizing the one or more fine-grained Gleason patterns in the input image.

At step 612, the plurality of logits may be converted to its corresponding probability value. This may be done using the Sigmoid Activation Function.

At step 614, the fine-grained Gleason grade can be calculated, by a computing module 112, based on the corresponding probability value. This fine-grained Gleason grade can be the continuous fine-grained Gleason grade, final fine-grained Gleason grade, or the updated Gleason grade, which may be calculated using the formulas previously described herein.

The various actions in method 600 may be performed in the order presented, in a different order or simultaneously. Further, in some embodiments, some actions listed in FIG. 6 may be omitted.

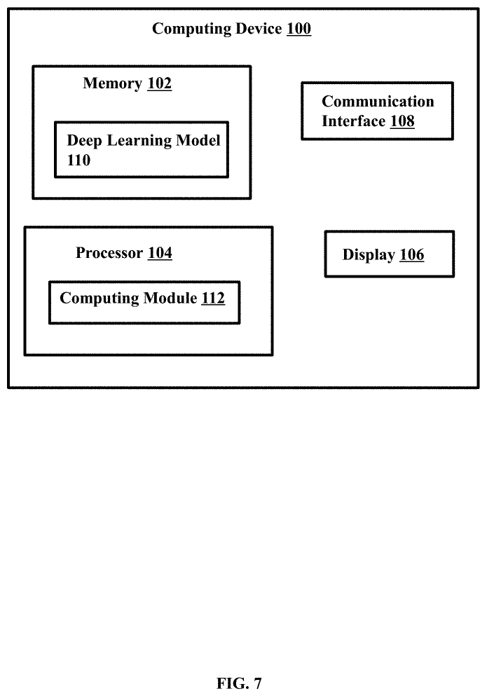

FIG. 7 illustrates a comparison of the results between a model using Cancer of the Prostate Risk Assessment Postsurgical (CAPRA-S) and a deep learning model 110 using fine-grained Gleason grades to determine the risk of metastasis, according to embodiments as disclosed herein. SegFormer was chosen as the segmentation network for the model being compared with the model using CAPRA-S. Based on the results for three cohorts of prostate cancer patients, the deep learning model 110 using fine-grained Gleason grades showed higher accuracy than the model using CAPRA-S in determining the risk of prostate cancer.

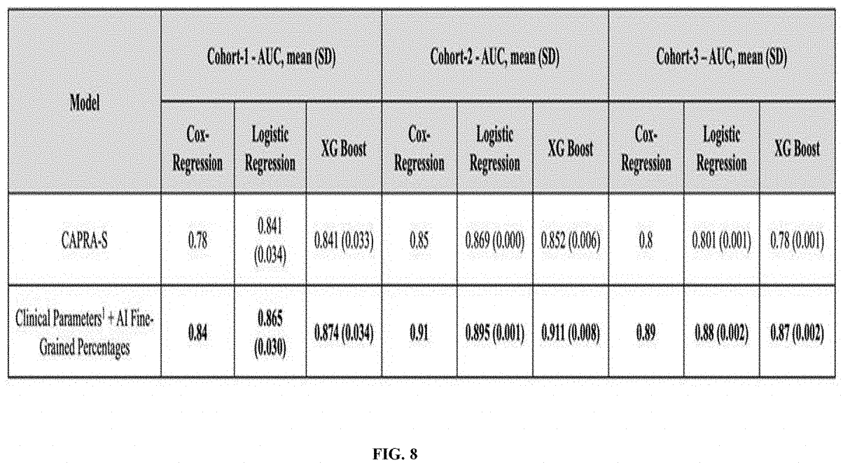

FIG. 8 illustrates a computing device 100 that is configured to produce the fine-grained Gleason grade, according to embodiments as disclosed herein. The computing device 100 may comprise a system having a memory 102 and a processor 104. The memory 102 may be able to store the training images that are used to train the deep learning model 110 to recognize fine-grained Gleason patterns in a RP image or needle biopsy image. The processor 104 (including the computing module 112) may be coupled to the memory, wherein the processor 104 is configured to perform the steps of method 600 upon executing a set of instructions stored in the memory 102. The computing module 112, may be configured to calculate the final fine-grained Gleason grade, the continuous fine-grained Gleason grade, and the upgraded Gleason grade.

The memory 102 may comprise, but is not limited to, volatile (e.g. random access memory (RAM)), non-volatile (e.g. read-only memory (ROM)), flash memory, or any combination. The processor 104 may represent one or more processors such as a microprocessor, a central processing unit or the like. The processor may also be a special-purpose processor such as an application specific integrated circuit (ASIC), a field programmable gate array (FPGA), a digital signal processor (DSP), a network processor, or the like. The computing device 100 may have its own display 106 such as a screen, or may be connected to an external display 106 to display the RP image and/or needle core biopsy image. The computing device 100 may receive images externally through a communication interface 108 that can facilitate communication with external devices using Wi-Fi, ZigBee, Bluetooth etc.

While the embodiments disclosed herein illustrate the usage of SegFormer as the deep learning model 110, a person skilled in the art may infer how to use other deep learning models to achieve fine-grained Gleason grades.

The embodiments disclosed herein describes systems and methods for producing a fine-grained Gleason grade. Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device. The method is implemented in at least one embodiment through or together with a software program written in e.g. Very high speed integrated circuit Hardware Description Language (VHDL) another programming language, or implemented by one or more VHDL or several software modules being executed on at least one hardware device. The hardware device can be any kind of portable device that can be programmed. The device may also include means which could be e.g. hardware means like e.g. an ASIC, or a combination of hardware and software means, e.g. an ASIC and an FPGA, or at least one microprocessor and at least one memory with software modules located therein. The method embodiments described herein could be implemented partly in hardware and partly in software. Alternatively, the invention may be implemented on different hardware devices, e.g. using a plurality of CPUs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein can be practiced with modification within the spirit and scope of the embodiments as described herein.

We claim:

1. A method for predicting prostate cancer risk based on a fine-grained Gleason grade, comprising:
receiving, by a deep learning model (110), an input image of a prostate tissue (input image);
analyzing, by the deep learning model (110), each pixel of the input image to recognize one or more fine-grained Gleason patterns;
outputting, by deep learning model (110), a plurality of logits on recognition of the one or more fine-grained Gleason patterns;
converting, by the deep learning model (110), the plurality of logits to its corresponding probability values using an activation function; and
calculating, by a computing module (112), the fine-grained Gleason grade based on the corresponding probability values, wherein the fine-grained Gleason grade is representative of the prostate cancer risk.

2. The method of claim 1, wherein the deep learning model (110) was trained to recognize the one or more fine-grained Gleason patterns using a training dataset comprising prostate tissue images with annotation masks.

3. The method of claim 1, wherein the fine-grained Gleason grade has at least one decimal place, or is rounded up or down to its integer value.

4. The method of claim 1, wherein the deep learning model (110) is SegFormer.

5. The method of claim 1, wherein the activation function is Sigmoid Activation Function.

6. The method of claim 2, wherein the prostate tissue images include at least one of: a needle core biopsy image and a radical prostatectomy image.

7. An apparatus (100) for predicting prostate cancer risk based on a fine-grained Gleason grade:
a memory (102), wherein the memory (102) is configured to store:
an input image of a prostate tissue (input image); and
a set of instructions; and
at least one processor (104), wherein the at least one processor (104) is configured to execute the set of instructions to result in the performance of at least one of the following:
receive the input image;
analyze each pixel of the input image to recognize one or more fine-grained Gleason patterns; and
output a plurality of logits on recognition of the one or more fine-grained Gleason patterns;
convert the plurality of logits to its corresponding probability values using an activation function; and
calculate the fine-grained Gleason grade based on the corresponding probability values, wherein the fine-grained Gleason grade is representative of the prostate cancer risk.

8. The apparatus (100) of claim 7, wherein the fine-grained Gleason grade has at least one decimal place, or is rounded up or down to its integer value.

9. The apparatus (100) of claim 7, wherein the input image includes at least one of: a needle core biopsy image and a radical prostatectomy image.
